(19) United States
(12) Patent Application Publication
(10) Pub. No.: US 2008/0182290 A1
(43) Pub. Date: Jul. 31, 2008

(54) Apparatus and methods for determining viability of cell-based products

(76) Inventor: Moshe Flugelman, Haifa (IL)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/901,560

(22) Filed: Sep. 17, 2007

Related U.S. Application Data
(60) Provisional application No. 60/844,908, filed on Sep. 15, 2006.

(51) Int. Cl. C12Q 1/04; C12M 1/00

(52) U.S. Cl. 435/34; 435/287.1; 435/288.1; 435/287.2

(57) ABSTRACT

The invention described herein pertains to an apparatus and methods for testing cell-based products, and more particularly to automated machinery that integrates various functions to determine cell viability at point of care locations.

### RELATED APPLICATIONS

This application claims priority to, and the benefit of, U.S. Provisional Application No. 60/844,908, filed Sep. 15, 2006, the contents of which are incorporated herein by reference in its entirety.

### FIELD OF THE INVENTION

The invention described herein pertains to an apparatus and methods for testing cell-based products, and more particularly to automated machinery that integrates various functions to determine cell viability at point of care locations.

### BACKGROUND

Cell therapy is the most challenging and rapidly developing discipline of modern medicine. The most pronounced use of cell therapy is bone marrow transplantation for hematological (blood) and cancer related syndromes. The range of diseases that are addressed with cell therapy is rapidly expanding to include autoimmune diseases and cardiovascular diseases. Additionally, the promise of stem cells (adult and embryonic) has captured the imagination of the scientific community and the public, and organ rejuvenation and cures for cancer are being tested with a variety of cells. Accordingly, thousands of medical centers around the globe use cell therapy for multiple indications.

The handling of the cell-based products and the manipulation that the cells undergo prior to transfer to a patient are unregulated and are at the discretion of the researcher. Currently, only a few countries regulate cell handling and processing. The major part of regulating cell-based products is quality control of the process and of the cell-based product. Since cells are dynamic and living entities, their condition can change rapidly. Biotech products in general, and cell-base products specifically, are highly sensitive to transfer and storage conditions.

Production of biotech products is usually done in a GMP facility under stringent conditions that include structured quality control and quality assurance processes. Before the final step of production and release of the product, the product is tested for several parameters such as sterility, or in the case of cell-based product, for cell viability. Once these tests are accomplished, the product is released for use. The final testing is referred to as “release testing.”

Biotech products are typically delivered to point of care facilities (e.g., hospitals or clinics), and may require special handling such as specific temperature and light conditions. These point of care facilities can be thousands of miles away from the manufacturing facility. Accordingly, even when the product is used immediately upon arrival (stem cell transplantation for example), or the facility and the patient are at the same hospital, unanticipated delays and/or exposure to extreme temperature can unknowingly damage or destroy the product. Currently, the point of care user (e.g., physician, nurse, or technician) and the patient have no convenient way of determining whether the quality and/or viability of the product has been compromised between the time the release testing was performed at the manufacturing facility and the time of use at the point of care. While several technologies exist that can test cell viability, none are equipped to do so simply, efficiently and with limited manipulation at the point of care.

Accordingly, it would be desirable to provide an apparatus and methods for performing point of care release testing just prior to the use of a cell based product to provide the user information regarding the quality and/or viability of the product.

### SUMMARY

The present invention provides an apparatus and methods for performing point of care release testing just prior to the use of a cell based product to provide the user information regarding the quality and/or viability of the product. The invention also provides an algorithm specific to different cell types wherein the algorithm is used to determine, based on the percentage of non-viable cells, whether or not the cell-based product should be used.

The invention includes an apparatus for performing point of care release testing of cell-based products. The apparatus can include means for thawing the cell based product, means for sampling the product, means for identifying viable or non-viable cells; and means for determining whether the product is useful for administering to a subject in need thereof.

The invention provides an apparatus for determining cell viability including at least one cell sampling chamber with a means for introducing one or more cells to the apparatus; at least one heating element disposed in operative proximity to the cell sampling system; at least one detection chamber in fluid communication with the cell sampling system; and an acquisition system for assessing the results obtained from the detection chamber, thereby determining the viability of the cells.

Suitable heating elements include a heat block, a hot plate, a water bath, a heating tank, and any combination thereof. Optionally, the heating element includes a means for agitating or mixing one or more cells contained within the cell sampling system.

The apparatus detection chamber includes a detection apparatus for evaluating the contents or characteristics of cells. Optionally, the detection apparatus comprises an optical or electrical detector. Suitable optical detectors include a spectrophotometer, a spectrometer, a spectrograph, a flow cytometer, and a fluorescence-activated cell sorter.

The apparatus can also include at least one labeling chamber with a means for labeling one or more cells. Optionally, the labeling chamber is in fluid communication with, and positioned between, the cell sampling system and the detection chamber. Optionally, the label is a protein, a DNA tag, a dye, an immuochemical agent, an antibody, an histochemical stain, a fluorescence probe, a quantum dot, radioactivity, a radio frequency identification tag, a change in viscosity, a change in opacity, a change in volume, a change in density, a change in pH, a change in temperature, a change in dielectric constant, a change in conductivity, or the change in the amount of any measurable entity within the one or more cells, or combinations thereof. The label can be detected by fluorescence polarization, fluorescence intensity, fluorescence lifetime, fluorescence energy transfer, pH, ionic content, temperature, or combinations thereof. The label identifies live cells, dead cells, or both.

The invention also provides for an apparatus that includes and acquisition system for assessing the results obtained from the detection chamber. Preferably, the acquisition system includes a microprocessor. The apparatus can include an output device for displaying cell viability data wherein the device is in communication with the acquisition system.

The means for introducing one or more cells to the apparatus can be a vacuum system, an injection system or a sliding piston. Optionally, the apparatus is automated.

The invention also provides methods for determining cell viability including the steps of: introducing one or more cells into a cell sampling chamber; heating the cells or cell suspension with a heating element; detecting the cells in a detection chamber; and assessing the results obtained from the detection chamber, thereby determining the viability of the cells. Preferably, the multiple cells are introduced to the cell campling chamber. More preferably, the cells are introduced as a cell population suspension. The cells can be detected with a spectrophotometer, a spectrometer, a spectrograph, a flow cytometer, or a fluorescence-activated cell sorter.

Optionally, the cells are labeled with a protein, a DNA tag, a dye, an immuochemical agent, an antibody, an histochemical stain, a fluorescence probe, a quantum dot, radioactivity, a radio frequency identification tag, a change in viscosity, a change in opacity, a change in volume, a change in density, a change in pH, a change in temperature, a change in dielectric constant, a change in conductivity, or the change in the amount of any measurable entity within the cells, or combinations thereof.

The cells can be detected by fluorescence polarization, fluorescence intensity, fluorescence lifetime, fluorescence energy transfer, pH, ionic content, temperature, or combinations thereof. The label identifies live cells, dead cells or both. Preferably, the results obtained from the detection system are assessed with a microprocessor.

The invention also includes a method for performing a point of care release test of a cell-based product by thawing the product, sampling the cell-based product in a sterile manner, determining viability of the cells, and determining if the cells are or are not suitable for use.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description.

### BRIEF DESCRIPTION OF DRAWINGS

FIG. 1 is a graphic representation showing cell staining with FACS.

FIG. 2 is a flow chart schematic illustrating the apparatus operation.

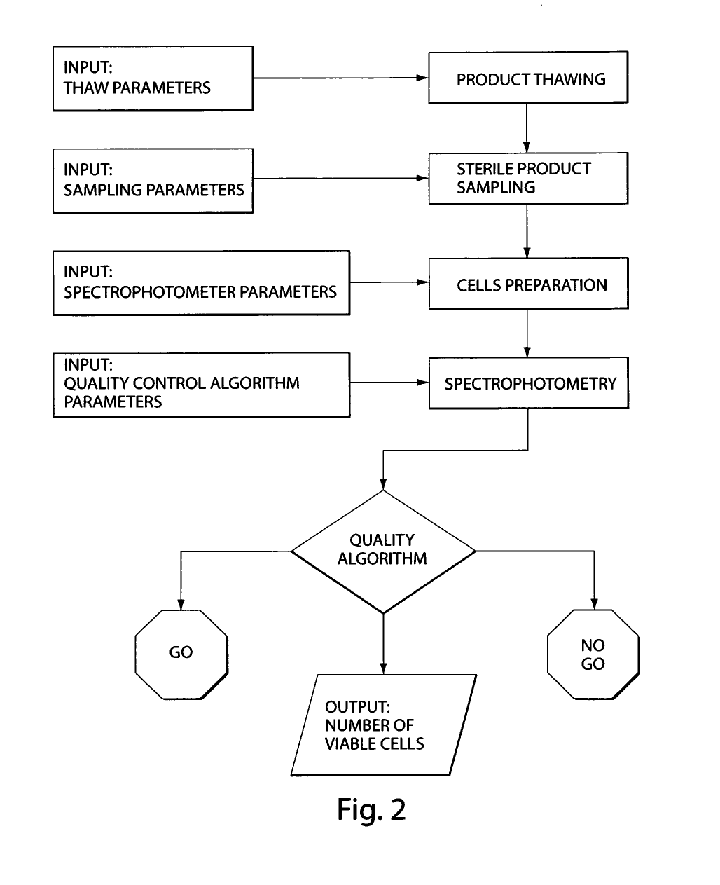

FIG. 3 is a flow chart schematic illustrating a preferred apparatus of the invention.

### DETAILED DESCRIPTION

The implications of point of care release testing technology have a huge impact on the business model of cell therapy companies. The shelf-life of a cell-based product can be very short. By providing the invention disclosed herein based on point of care release testing of cell-based products (positioned for example in the operating room), the product can be delivered from the manufacturer frozen on dry ice and then thawed by the point of care machine, tested for cell viability and programmed to indicate use/no use of the cell-based product based on the test for cell viability. In case of delays, the test can be repeated prior to use of the cell-based product. Accordingly, the distance from the manufacturing facility to the point of care facility will no longer be a limiting factor. Further, in view of the apparatus and methods provided herein, special laboratories in the point of care facility to test the product after transportation and thawing will not be required.

The invention includes an apparatus for performing point of care release testing of cell-based products. In an exemplary embodiment, the apparatus includes means for thawing the product, means for sampling the product, means for identifying viable or non-viable cells, and means for determining whether the product is useful for administering to a subject in need thereof.

The term “viable” refers to cells that maintain homeostasis by the use of one or more energy consuming mechanisms. Thus a “viable” cell includes those in which productive oxidative metabolism occurs to produce the necessary energy; those in which only glycolysis is used to produce energy, as well as those which maintain cellular integrity, such as the ability to exclude, or actively remove, certain molecules from the interior of the cell, by energy consuming mechanisms. Preferably, a “viable” cell is capable of undergoing mitosis, cell growth, differentiation, and/or proliferation. A “viable” cell is synonymous with a “living” cell, which includes cells that are quiescent (and thus not going through the cell cycle), but nonetheless alive because energy production and consumption occurs in such cells to maintain homeostasis.

Evaluation of cell viability is important for assessing the effect of drugs, environmental pollutants, irradiation, temperature, ionic extremes, and other potential biological modifiers. Traditionally, cell membrane integrity is used as an indicator of cell viability, as damage to the protective cell membrane often results in loss of cell structure, leakage of critical intracellular contents, breakdown of essential ionic gradients and ultimately cell death. Another indicator of cell viability is intracellular activity, the presence of which activity indicates that the cell is able to metabolize, grow, reproduce, maintain electrical membrane potential, or perform some other cell function critical for viability. Conversely, the lack of such activity is often used as an indicator of cell death.

The viability of a cell-based product in a test sample can be determined in order to assess the quality of the cell-based product. Suitable instruments for determining cell viability include a fluorescence-activated cell sorter (FACS), a spectrophotometer, a spectrometer, a spectrograph, and a pH meter. Alternatively, any standard method for determining cell viability can be utilized.

This invention also provides an apparatus for performing analytical analysis of samples, preferably biological samples. A biological sample means any substance isolated from or derived from any organism. A biological sample can be tissue, blood, prokaryotic cells, eukaryotic cells, cell lines, cell organelles, antibodies, hybridomas, plasmids, viruses, plant tissue cells, bacteria, fungi including yeast, algae, protozoa, lichens, seeds, viruses or vectors. Preferably, the biological sample is a cell based product comprising one or more cells. Sterile samples of the cell-based product are collected to avoid contamination of the cells. For the purposes of this invention, the term “sample” will be understood to encompass any fluid containing a particulate species of interest, wherein the particulate species is preferably a cell. Any type of cell is useful in the methods described herein. Suitable samples include cells such as blood cells, lymphocytes, and cells intended for use in cell therapy, which is the process of introducing new cells into a tissue in order to treat a disease. Suitable samples can be isolated from any animal or mammalian source. Preferably, the samples are isolated from a human and preferably the samples are for human use (e.g., therapeutic or prophylactic treatment). The following properties or applications of these methods will essentially be described for humans although they may also be applied to non-human mammals, e.g., apes, monkeys, dogs, mice, etc. The invention therefore can also be used in a plant or veterinarian context. Cells useful in the invention include one or more of the following: hematopoietic stem cells, endothelial stem cells, hepatic stem cells, neuronal stem cells, muscle stem cells, cardiac stem cells, adult stem cells, embryonic stem cells, epidermal stem cells, adipose stem cells, mesenchymal stem cells, epithelial stem cells, stem cells obtained from a zygote, stem cells obtained from a blastocyst, stem cells from any organ, stem cells from any tissue, neurons, oligodendrocytes, astrocytes, smooth muscle cells, endothelial cells, cells from any organ (brain, pancreas, liver, kidney, heart, etc.), cells from any tissue (spinal cord, muscle, upper and lower gastrointestinal tracts, etc.) and combinations thereof. Other suitable cells include both autologous (from the patient) and allogeneic (from another donor) stem cells, and cells that are genetically modified to express or over-express a gene or protein of interest. Optionally, the sample is maintained in a cultured medium.

For use in the apparatus and methods of this invention, the cells are optionally in suspension or are immobilized on a solid or semisolid support. The cells can be in suspension on a microscope slide or in a specialized container needed for an instrumentation detection method such as in a cuvette or in a microtiter plate (e.g. 96 well titer plate). Alternatively, the cells are adhered to a microscope slide using a cell adhesive solution such as poly-L-lysine, or are attached to a filter as a retained residue.

Optionally, the apparatus of the invention further comprises a sample input means, preferably comprising metering elements to deliver a volumetric amount of sample fluid to the cell sampling chamber of the apparatus. Preferably, the means for introducing the cells to the apparatus is a vacuum system, an injection system, or a sliding piston. The apparatus of the invention can also comprise an overflow reservoir for retaining excess fluid applied to the apparatus in excess of the amount metered into the cell sampling chamber, most preferably in fluid communication with the fluid sample input means wherein excess fluid is transferred to the overflow reservoir. The overflow chamber is connected to the entry port to take off any excess fluid. For the purposes of this invention, the term “in fluid communication” or “fluidly connected” is intended to define components that are operably interconnected to allow fluid flow between components.

Advantageous components of the apparatus of the invention include fluid sample input means, including volumetric metering means, channels for fluid flow between components, reagent reservoirs, mixing chambers, optical or electrical reading chambers, and filtering means that retain cells in the chamber. The invention can also provide valves for controlling fluid flow between components, temperature control elements, separation channels, air outlet ports, sample outlet ports, mixing means including magnetic, acoustic and preferably mechanical mixers, liquid and dry reagents, and other components as described herein or known to the skilled artisan.

The fluid flow in the apparatus of the invention can be provided by mechanical means, including but not limited to using pumping means sufficient to achieve fluid movement. These means might include syringe pumps or HPLC pumps. The fluid flow in the apparatus can also be provided by electrical, osmotic, electroosmotic means or any means know in the art for achieving fluid movement.

The sample is applied to the cell sampling chamber of the apparatus of the invention either directly or more preferably by transfer of a metered amount of a portion of the sample from a fluid sample input means to the chamber, for example, by the selective opening of valves controlling access to the chamber from the fluid sample input means. The valves include, but are not limited to microvalves including mechanical, electrical and thermal valve mechanisms. Reagent reservoirs, wash buffer reservoirs, other fluidic components and the contents thereof are connected to one another and to the detection and cell accumulation chambers through channels, preferably microchannels as defined herein, controlled by such valves.

The invention also provides an apparatus that contains reservoirs and chambers for containing fluids, such as a wash buffer and staining solution. Optionally, the apparatus contains washing buffer used to detach nonspecifically bound particulates from the surface of the detection chamber, or a solution of a compound to which cells in the cell sampling chamber are going to be exposed. The chambers may be pre-filled with liquid components and sealed using valving mechanisms, may be filled with dried reagents which are resolubilized by the addition of a fluid such as water, or may be filled at the time of use with prepared liquid reagents.

The cell based product can be delivered from the manufacturer at any temperature. The cell based product is often delivered at less than about room temperature, at less about 4° C., at about 0° C. or less than about −4° C. Optionally, the sample of the cell-based product is delivered from the manufacturer frozen on dry ice or frozen in liquid nitrogen. The apparatus includes at least one cell thawing/heating element in operative proximity to the cell sampling system. The means for heating or thawing cells includes a heat block, a hot plate, a water bath, a heating tank, or any combination thereof. Optionally, the heating element includes a means for agitating or mixing one or more cells contained within the cell sampling system.

The invention also provide at least one labeling chamber comprising a means for labeling the cells, wherein the labeling chamber is in fluid communication with, the cell sampling chamber and the detection chamber. Stains and/or dyes are used to assess the viability of cells. Although a single stain/dye can be used to assess viability, the use of a combination of dyes has advantages. First, the use of a dye combination allows one skilled in the art to determine the ratio of the number of cells that show a response to the one dye versus the total number of cells or versus those cells that do not respond. Optionally, a second dye is used as a positive control to indicate that other cells are present that did not stain with the first dye.

Several methods using a combination of fluorescent dyes for the analysis of cell viability have been developed, including methods that use differential fluorescent staining of live and dead cells. See, Haugland, Handbook Of Fluorescent Probes And Research Chemicals Sets 25 & 31 (1992). Live, intact cells can generally be distinguished from dead cells with compromised membranes by differential staining using a cell-impermeant fluorescent dye that only enters dead cells, and a cell-permeant dye that enters both live and dead cells but requires intracellular activity indicative of viability for the production of fluorescence. Alternatively, differential fluorescent staining can involve the use of two cell-permeant dyes where one stains both live and dead cells and the other stains cells only when an intracellular reaction produces fluorescence.

Optionally, the fluorescent dye passes through damaged cell membranes or the cell membrane of dead cells, but is unable to pass through undamaged cell membranes or the cell membrane of viable cells. Therefore, when the fluorescent probe is applied to a test sample in which both viable and dead cells are coexisting, the fluorescent probe selectively permeates into the dead cells and bonds with nucleic acids to emit a strong fluorescence. The dead cell numbers are determined by measuring the fluorescence intensity with a fluorometer. The fluorescent probe can include cationic fluorescent stains (dyes) for nucleic acids, such as ethidium homodimer and a cyanine fluorescent dye; ethidium halides such as ethidium bromide; propidium halides such as propidium iodide; and the like.

The dye solution or staining solution can be made by dissolving the dye directly in an aqueous solvent such as water, a buffer solution, such as phosphate buffered saline, or an organic water-miscible solvent such as dimethylsulfoxide (DMSO), dimethylformamide (DMF), or a lower alcohol such as methanol or ethanol, or acetonitrile. Dyes that possess low water solubility are typically preliminarily dissolved in an organic solvent (preferably DMSO) at a concentration of greater than about 100-times that used in the staining solution, then diluted one or more times with an aqueous solvent such as water or buffer. Preferably, the dye is dissolved in about 100% DMSO and then diluted one or more times in water or buffer such that the dye is present in an effective amount. An effective amount of dye is the amount sufficient to give a detectable fluorescent response in the cells being analyzed. Typically the concentration of the dye is between about 0.01 μm and about 100 μM. It is generally understood in the art that the specific concentration of the staining solution is determined by the physical nature of the sample.

The optimal concentration of the dye is generally determined according to the cell density. A range of dye concentrations are used to stain the sample or cell suspensions to determine the optimal dye concentration for the cell density of the sample. Typically, dye concentrations from about 1 mM down are tested, preferably dye concentrations from about 30 μM down to about 1.1 μM. The tested ranges of dye concentration represent the ranges used for the analysis.

Following preparation of the dyed cells, the cells are illuminated at a suitable absorption wavelength. A suitable wavelength is one that comes within the range of absorption wavelengths for each of the fluorescent dyes being used. Typically, the mixture is illuminated by a light source capable of producing light at or near the wavelength of maximum absorption of the dye or dyes, such as by ultraviolet or visible lamp, an arc lamp, a laser, or even sunlight. Illumination of the dyed cells at a suitable wavelength results in one or more illuminated cells that are then analyzed according to their fluorescent response to the illumination.

One of the main features separating dead from live cells is the loss of the physical integrity of the plasma membrane (Darzynkiewicz, Z., Li, X., and Gong, J. P. (1994) in Methods in Cell Biology Academic Press, Inc., New York; King, M. A. (2000) J. Immunol. Methods 243, 3-12.) When the membrane integrity is lost, chemicals that would otherwise not enter the cell can enter. Therefore, a variety of viability tests have been designed which test if chemicals that cannot penetrate the membrane of intact cells, are inside the cells. The most common of such chemicals are colorimetric dyes such as trypan blue and fluorescent dyes such as propidium iodide or YOYO-1 (Molecular Probes OR), which change the cell color once inside the cell (Horan, P. K., and Kapler, J. W. (1977) J. Immunol Methods 18, 309-316; Shapiro, H. M. (1995) in Practical Flow Cytometry Wiley, New York; Haugland, R. P. (1996) Handbook of Fluorescent Probes and Research Chemicals, Molecular Probes, Inc.) These chemicals are commonly used to determine cell viability in cells in suspension (Rui, J., Tatsutani, K. N., Dahiya, R., Rubinsky, B. Effect of thermal variables on human breast cancer in cryosurgery. Breast Cancer Research and Treatment, 53 182-192, 1999) as well as cells in tissue that was excised from the body (Pham, L., Rubinsky, B., “Breast tissue cryosurgery with antifreeze proteins” HTD-Vol. 362/BED-Vol. 40, Advances in Heat and Mass Transfer in Biotechnology—ASME Press, pp 171-175. 1998).

The apparatus of the invention also provides a detection chamber for evaluating at least one content or characteristic of the cells comprised in the cell based product. The invention includes detection systems for detecting, monitoring, quantifying or analyzing particulates, such as cells, in a detection chamber or in a cell accumulation chamber as described herein.

The illuminated cells are observed with any of a number of means for detecting a fluorescent response emitted from the illuminated cells, including but not limited to visible inspection, cameras and film or other imaging equipment, or use of instrumentation such as fluorometers, plate readers, laser scanners, microscopes, or flow cytometers, or by means for amplifying the signal such as a photomultiplier.

Detection systems useful in the manufacture and use of the apparatus of the invention include, but are not limited to, fluorescent, chemiluminescent, calorimetric, or scattering measurements. The detection chamber or cell accumulation chamber can constitute a cuvette that is illuminated.

The detection apparatus can comprise an optical or electrical detector. Preferably, the detection apparatus comprises an optical detecting means. Optionally, the detection system comprises a simple visual detection means such as the development of a visible color. Alternatively, non-optical detection systems such as electrochemical and radioactivity detecting are used.

Optical detecting means are provided as components of the apparatus of the invention. The photodetectors of the invention are optimally provided to detect optical absorbance/transmittance, fluorescence, light-scattering or other optical signals, which are processed and translated into data on the position, number and viability of cells.

Absorbance measurements can be used to detect a dye or stain, such as a vital stain, or other analyte that changes the intensity of transmitted light by specifically absorbing energy (direct absorbance) or by changing the absorbance of another component in the system (indirect absorbance). Absorbance measurements are preferably used in conjunction with enzyme-linked detection of the presence of a particulate within a detection chamber. In preferred embodiments, cellular particulates are detected by vital staining or other cell-specific staining (such as the use of dyes specific for certain cell types). Optical path geometry is designed to ensure that the absorbance detector is focused on a light path receiving the maximum amount of transmitted light from the illuminated sample.

The invention provides an optical detector such as a spectrometer, a spectrograph, a flow cytometer, or a fluorescence-activated cell sorter (FACS). Detection systems for use in the invention include spectroscopic, particularly monochromatic and stroboscopic, and electrochemical detectors (see, for example, Owicki et al., 1992, Biosensors & Biolelectronics 7: 255). Spectroscopic methods using these detectors encompass spectroscopy, particularly ultraviolet and visible light absorbance, chemiluminescence, and fluorescence spectroscopy. Generally, the detection systems of the invention comprise a light source and a photodetector; in certain embodiments (such as chemiluminescence), only the photodetector components are required.

Preferred spectroscopic methods include fluorescence. For example, an excitation source such as a laser is focused on an optically-transparent section of a disk. Light from any analytically-useful portion of the electromagnetic spectrum is used to illuminate a particulate retained in an detection chamber or cell incubation chamber or surface. Alternatively, the selection of light at a particular wavelength is paired with a material having geometries and refractive index properties resulting in total internal reflection of the illuminating light. This enables either detection of material on the surface of the disk through evanescent light propagation, or multiple reflections through the sample itself, which increases the path length considerably.

Fluorescence activated cell sorting (FACS) is a powerful method that has been used to identify cells having a particular phenotype. See Herzenberg et al. (Clincal Chem. 48(10):1819-1827 (2002)) for a review. In some forms, the FACS method has been used in combination with monoclonal antibodies as a reagent to detect cells as having a particular antigen, which is usually indicative of an expressed protein. The method has been used extensively in relation to antigens expressed on the surface of cells, including cells that remain alive during, and after, FACS. Similarly, the method has been used with intracellular reporter gene systems based on the expression of a detectably labeled gene product by the cell.

Optionally, the determination of viability can be used as a basis for sorting the cells for further experimentation. For example, all viable cells in a population are sorted, or all dead cells in a population are sorted. The cells are sorted manually or using an automated technique such as flow cytometry according to the procedures known in the art such as in U.S. Pat. No. 4,665,024

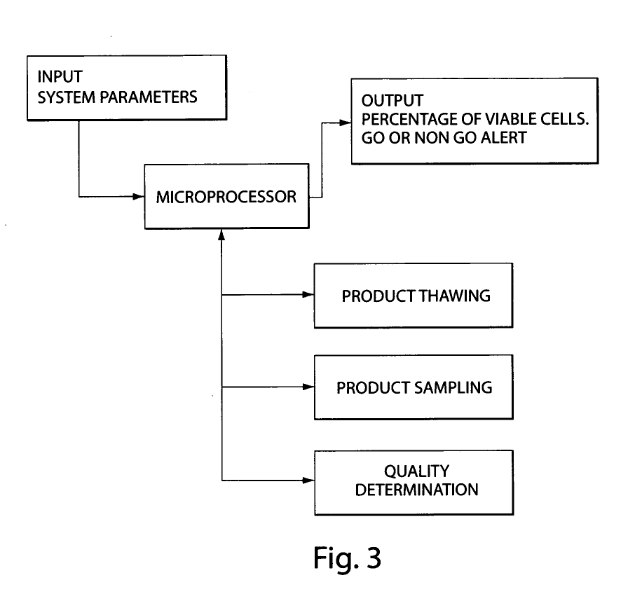

The cell viability can be calculated from the total cell number and viable cell number data obtained by the assay. Preferably, the invention includes a microprocessor. The microprocessor of the invention is a programmable digital electronic component that incorporates the functions of a central processing unit (CPU) on a semiconducting integrated circuit (IC). As shown in FIG. 3, the microprocessor receives input for the various steps of determining cell viability; controls the process of determining cell viability; runs the algorithm of quality determination; and displays the result to the user. Additionally, the microprocessor saves the history of the process for further statistical analysis.

A user interface (e.g., computer), including keypads, light-pens, monitors, indicators, flat-panel displays, interface through communications options to host-devices or peripheral devices, and printers, plotters, and graphics devices are provided as devices of the invention. Communication and telecommunications are provided through standard hard-wired interfaces (such as RS-232, IEEE-488M SCSI bus), infra-red and optical communications, short- or long-range telecommunications (“cellular” telecommunications radio-frequency), and internal or external modem for manual or automated telephone communications.

The apparatus of the invention determines the viability of the cells and if the cells are or are not suitable for use. The microprocessor runs an algorithm designed to determine the product quality (cell viability). The following factors are taken into consideration when determining product quality: the type of cells, the input of the measured parameters, and the appropriate permitted range. The apparatus displays a GO or NO GO output to the user indicating if the product can or cannot be used. In some embodiments, a GO output is displayed when at least 50% of the cells sampled in the cell based product are viable. In other embodiments, the GO output is displayed when at least 60%, at least 70%, at least 80% or at least 90% of the cells sampled in the cell based product are viable.

Preferably, the means for thawing can include a heating element integrated in the apparatus as part of a heat block or water bath. The means for testing the product can include a FACS or spectrophotometer, pH meter, or other parameters important for determining the quality or viability of the product. In another embodiment, the means for identifying viable or non-viable cells can include cell staining methods known by those skilled in the art. The means for determining whether or not to use the cell-based product can include an algorithm based on the number of viable or non-viable cells in the product, pH, or other relevant parameters. The apparatus can also include means for displaying a message to the use indicating whether or not the product is suitable based on the outcome of the algorithm.

The invention can include a method for performing a point of care release test of a cell-based product by thawing the product, sampling it in a sterile manner, determining viability of the cells, and determining use or non-use of the cells.

The temperature of the cell-based can be manipulated to the appropriate utilization temperature prior to testing. Preferably, the cells are heated or thawed to room temperature or about 37° C. Suitable thawing means include a controllable hot plate, a water bath and a tank containing an agitating frame. The microprocessor determines the appropriate thawing protocol according to quantity and type of cells.

Samples of the cell-based product are examined to determine cell viability. Sterile samples are collected to avoid contamination of the cells. Optionally, the apparatus includes a vacuum system or a sliding piston to extract the solution from the container. Alternatively, the apparatus uses any other standard method to extract the solution from the container. The microprocessor controls each of the sampling phases and determines (accord to the respective input) the amount of material to be sampled.

By way of non-limiting example, a cell-based product can be transferred from the cell processing/manufacturing facility to the point of care facility in dry ice. Using the device and methods described herein, the cells can be thawed at a pace and time that were predetermined for the specific cell type by the manufacturer.

After thawing, the apparatus can sample the product via a specific port in the product container and can stain the cell in the sample with a specific dye that is used to identify dead cells. The stained sample can pass through the spectrophotometer which is part of the apparatus and based on the reading of the spectrophotometer, the percentage of cells that are dead can be determined. The apparatus can use the percentage of dead cells in an algorithm and can display a message to the user whether the product is suitable or not for administration to a patient in need thereof.

Without further elaboration, it is believed that one skilled in the art can, using the preceding description, utilize the present invention to its fullest extent. The following example is, therefore, to be construed as merely illustrative, and not limitative of the remainder of the disclosure in any way whatsoever.

### EXAMPLE 1

### FACS Analysis of Cell Viability

The viability of a test sample of cells was determined using a fluorescence-activated cell sorter (FACS). The red stain component of the LIVE/DEAD® Reduced Biohazard Viability/Cytotoxicity Kit #1 (Molecular Probes, cat# L-7013) was used for cell viability testing.

The basis for this viability test was the differential permeability of live and dead cells to fluorescent stains. The cell-impermeant, red fluorescent nucleic acid stain labels only cells with compromised membranes.

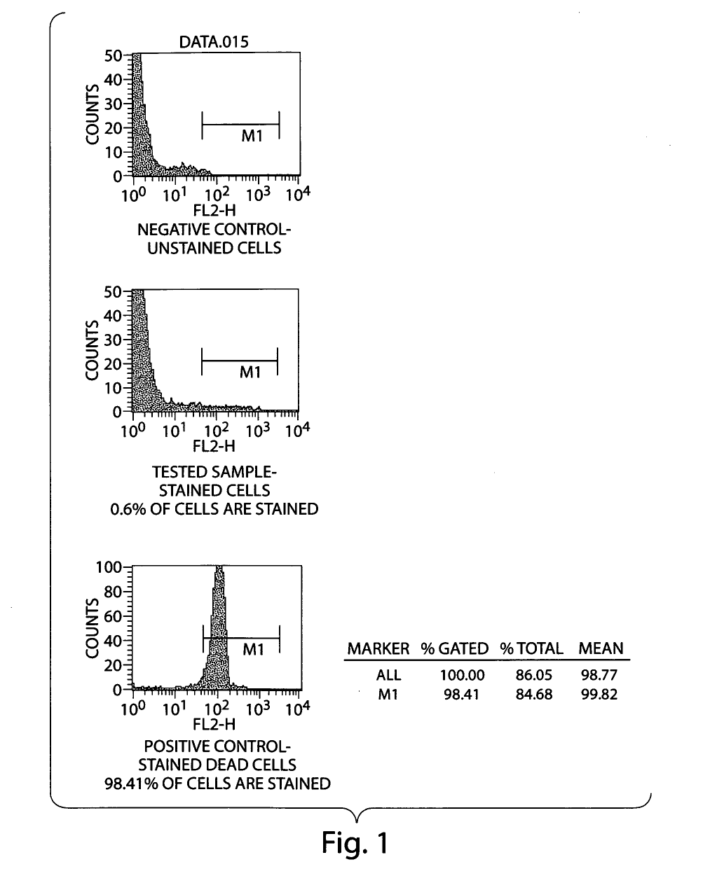

Cells (10⁵) were taken from a test sample and incubated for 15 min with a commercial reagent that contains the two above stains. MGA cells incubated without the red stain served as negative control. MGA cells pre-treated with 50% alcohol in order to permeabilize the membranes served as positive control. Following incubation, the cells were analyzed via FACS. The percentage of live and dead cells in each group was determined (FIG. 1).

What is claimed is:

1. An apparatus for determining cell viability comprising:
a) at least one cell sampling chamber comprising a means for introducing one or more cells to the apparatus;
b) at least one heating element disposed in operative proximity to said cell sampling system;
c) at least one detection chamber in fluid communication with said cell sampling system; and
d) an acquisition system for assessing the results obtained from the detection chamber,
thereby determining the viability of said cells.

2. The apparatus of claim 1, wherein said heating element is a heat block, a hot plate, a water bath, a heating tank, or any combination thereof.

3. The apparatus of claim 2, wherein said heating element comprises a means for agitating or mixing one or more cells contained within said cell sampling system.

4. The apparatus of claim 1, wherein said detection chamber comprises a detection apparatus for evaluating the contents or characteristics of said one or more cells

5. The apparatus of claim 2, wherein said detection apparatus comprises an optical or electrical detector.

6. The apparatus of claim 5, wherein said optical detector comprises a spectrometer, a spectrograph, a flow cytometer, or a fluorescence-activated cell sorter.

7. The apparatus of claim 1, wherein said apparatus further comprises at least one labeling chamber comprising a means for labeling said one or more cells, wherein said labeling chamber is in fluid communication with, and positioned between, said cell sampling system and said detection chamber.

8. The apparatus of claim 7, wherein said label is a protein, a DNA tag, a dye, an immuochemical agent, an antibody, an histochemical stain, a fluorescence probe, a quantum dot, radioactivity, a radio frequency identification tag, a change in viscosity, a change in opacity, a change in volume, a change in density, a change in pH, a change in temperature, a change in dielectric constant, a change in conductivity, or the change in the amount of any measurable entity within said one or more cells, or combinations thereof.

9. The apparatus of claim 8, wherein said label is detected by fluorescence polarization, fluorescence intensity, fluorescence lifetime, fluorescence energy transfer, pH, ionic content, temperature, or combinations thereof.

10. The apparatus of claim 9, wherein said label identifies live or dead cells.

11. The apparatus of claim 1, wherein the acquisition system is a microprocessor.

12. The apparatus of claim 1, further comprising an output device for displaying cell viability data wherein said device is in communication with the acquisition system.

13. The apparatus of claim 1, wherein said means for introducing one or more cells to the apparatus is a vacuum system, an injection system or a sliding piston.

14. The apparatus of claim 1, wherein said apparatus is automated.

15. A method for determining cell viability comprising the steps of:
a) introducing one or more cells into a cell sampling chamber;
b) heating said one or more cells with a heating element;
c) detecting said one or more cells in a detection chamber; and
d) assessing the results obtained from said detection chamber, thereby determining the viability of said cells.

16. The method of claim 15, wherein said one or more cells are detected with a spectrometer, a spectrograph, a flow cytometer, or a fluorescence-activated cell sorter.

17. The method of claim 15, wherein said one or more cells are labeled with a protein, a DNA tag, a dye, an immuochemical agent, an antibody, an histochemical stain, a fluorescence probe, a quantum dot, radioactivity, a radio frequency identification tag, a change in viscosity, a change in opacity, a change in volume, a change in density, a change in pH, a change in temperature, a change in dielectric constant, a change in conductivity, or the change in the amount of any measurable entity within said one or more cells, or combinations thereof.

18. The method of claim 17, wherein said label is detected by fluorescence polarization, fluorescence intensity, fluorescence lifetime, fluorescence energy transfer, pH, ionic content, temperature, or combinations thereof.

19. The method of claim 18, wherein said label identifies live or dead cells.

20. The method of claim 15, wherein said results obtained from said detection chamber are assessed with a microprocessor.
